(19) United States
(12) Patent Application Publication
(10) Pub. No.: US 2017/0224197 A1
(43) Pub. Date: Aug. 10, 2017

(54) ENDOSCOPE SYSTEM INCLUDING DRIP RETENTION FEATURES

(71) Applicant: GYRUS ACMI, INC., d/b/a Olympus Surgical Technologies America, Southborough, MA (US)

(72) Inventors: Joseph Green, Bartlett, TN (US); Joel Andrew Willhite, Memphis, TN (US)

(21) Appl. No.: 15/414,998

(22) Filed: Jan. 25, 2017

Related U.S. Application Data
(60) Provisional application No. 62/291,728, filed on Feb. 5, 2016.

(51) Int. Cl. A61B 1/00; A61B 1/12; A61B 1/015

(52) U.S. Cl. CPC A61B 1/00135; A61B 1/015; A61B 1/126

(57) ABSTRACT

An endoscope sheath comprising: (a) a proximal end, (b) a distal end having a distal end region, (c) a surface extending between and connecting the proximal end and the distal end, (d) a plurality of positioning devices located along the surface; and (e) one or more drip retention features; wherein the sheath is configured to: (i) receive all or a portion of an endoscope and (ii) provide a conduit for communicating fluid between the proximal end of the endoscope sheath and the distal end of the endoscope sheath when the endoscope is inserted inside the sheath; and wherein the plurality of positioning devices are located in a distal end region at the distal end of the endoscope sheath so that the endoscope is secured within the endoscope sheath; and wherein the one or more drip retention features are configured to produce a region bounded by the endoscope sheath and the endoscope to retain a volume of liquid by capillary action.

### FIELD

The present teachings generally relate to a system including an endoscope sheath that assists in washing a lens and/or imaging device of an endoscope and preventing fluid and/or drips from forming and/or being retained on the lens and/or imaging device that distort a user's vision.

### BACKGROUND

Endoscopes are typically used for minimally invasive surgery or to provide access to an internal location of a patient so that a doctor is provided with visual access. Endoscopes, during use, may be inserted into a location that may include debris that may cover the end of the endoscope and especially cover an imaging device located at the end of the endoscope such that imaging is obscured. For example, an endoscope being used for surgery may become covered by blood and the blood may impair the vision of a surgeon so that surgery becomes increasingly difficult. Attempts have been made to provide various devices and systems to assist a surgeon in clearing debris from the imaging device of the endoscope and restore vision. These devices may remove some of the debris from the imaging device of the endoscope, however, these devices may not remove all of the debris and/or may leave spots or droplets on the imaging device, which may result in continued impairment. These devices may have features that attempt to control the flow of fluid, suction, or both at the end of the endoscope in an attempt to clear debris, spots, droplets, or a combination thereof from the endoscope. Further, some of the endoscope systems leave droplets on the imaging device and/or lens and these droplets may inhibit vision through the lens and/or imaging device. The application of irrigation fluid and suction may clear debris and items that obstruct a view, but the irrigation fluid may remain on the lens or the imaging device even after the application of suction such that the irrigation fluid continues to obscure the vision of the surgeon.

Examples of some surgical instruments and endoscope cleaning systems may be found in U.S. Pat. Nos. 5,405,348; 5,575,756; 5,630,795; 7,063,112; and 8,677,843; U.S. Patent Application Publication Nos. 2015/0087907, 2015/0087909; and 2015/0087911 all of which are incorporated by reference herein in their entirety herein for all purposes. It would be attractive to have an endoscope system that washes an endoscope lens and/or imaging device and removes all debris and fluid droplets so that vision is not impaired. What is needed is an endoscope sheath that removes droplets from a lens and/or imaging device so that a surgeon's vision is not impaired. It would be attractive to have an endoscope sheath that retains a volume of liquid and removes fluid from the distal end of the endoscope. It would be attractive to have an endoscope sheath that is configured to retain a volume of liquid after irrigation fluid and suction are applied so that any remaining fluid is removed from the lens and/or imaging device.

### SUMMARY

The present teachings meet one or more of the present needs by providing: an endoscope sheath comprising: (a) a proximal end, (b) a distal end having a distal end region, (c) a surface extending between and connecting the proximal end and the distal end, (d) a plurality of positioning devices located along the surface; and (e) one or more drip retention features; wherein the sheath is configured to: (i) receive all or a portion of an endoscope and (ii) provide a conduit for communicating fluid between the proximal end of the endoscope sheath and the distal end of the endoscope sheath when the endoscope is inserted inside the sheath; and wherein the plurality of positioning devices are located in a distal end region at the distal end of the endoscope sheath so that the endoscope is secured within the endoscope sheath; and wherein the one or more drip retention features are configured to produce a region bounded by the endoscope sheath and the endoscope to retain a volume of liquid by capillary action.

The teachings herein provide an endoscope system that washes an endoscope lens and/or imaging device and removes all debris and fluid droplets so that vision is not impaired. The teachings herein provide an endoscope sheath that removes droplets from a lens and/or imaging device so that a surgeon's vision is not impaired. The teachings herein provide an endoscope sheath that retains a volume of liquid and removes fluid from the distal end of the endoscope. The present teachings provide an endoscope sheath that is configured to retain a volume of liquid after irrigation fluid and suction are applied so that any remaining fluid is removed from the lens and/or imaging device.

### BRIEF DESCRIPTION OF THE DRAWINGS

FIG. 1 illustrates a side perspective view of an endoscope sheath;

FIG. 2 illustrates a perspective view of a bottom of an endoscope sheath;

FIG. 3 illustrates a distal end view of an endoscope sheath including an endoscope;

FIG. 3A illustrates a close-up view of drip retention feature and an endoscope;

FIG. 3B illustrates a close-up view of a gap between a drip retention feature and an endoscope;

FIG. 3C illustrates a close-up partial cross-sectional view of a volume of fluid between the drip retention feature and endoscope;

FIG. 3D illustrates a close-up partial cross-sectional view of a volume of fluid in the gap between the drip retention feature and the endoscope;

FIG. 4 illustrates a distal end view of an endoscope sheath with the endoscope removed;

FIG. 5 illustrates a plan view of a bottom of an endoscope sheath;

FIG. 5A illustrates a close-up view of a drip retention feature of FIG. 5;

FIG. 6 illustrates a plan view of a top of an endoscope sheath;

FIG. 6A illustrates a close-up view of engravings of FIG. 6;

FIG. 7 illustrates a cross-sectional view of FIG. 5 along line 7-7;

FIG. 7A illustrates a close-up view of the drip retention feature of FIG. 7; and

FIG. 8 illustrates a system including the endoscope sheath.

### DETAILED DESCRIPTION

The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

The present application claims priority to U.S. Provisional Patent Application No. 62/291,728, filed Feb. 5, 2016. The present teachings provide an endoscope sheath for use in a system. The system of the teachings herein includes an irrigation source and a suction source that are both connected to an endoscope sheath and are in communication with an endoscope. The system may include one or more control modules. The system may function to clean an endoscope. Preferably, the system functions to clean a distal end (i.e., a tip) of an endoscope. More preferably, the system functions to clean a lens and/or an imaging device of an endoscope. The system may include one or more functional components that may extend proximate to a distal end of an endoscope or beyond a distal end of an endoscope. The system may provide one or more conduits relative to the endoscope. The system may protect the endoscope. The system as is taught herein may include a resilient reservoir or endoscope sheath as taught herein may be in communication with a resilient reservoir of the teachings herein including those found in U.S. Patent Application Publication No. 2015/0087909 in paragraph nos. 004-005; 020-065 including FIGS. 4-10, the teachings of which are all expressly incorporated by reference herein as to the system, resilient reservoir, and communication with the endoscope sheath. The system of the present teachings may include one or more of the positioning devices found herein including those found in U.S. Patent Application Publication No. 2015/0087907 in paragraph nos. 0062-0083 and FIGS. 4A-12C the teachings of which are expressly incorporated by reference herein as to the positioning devices and surrounding structure. The system may include one or more sources of irrigation fluid for use with the system, and the one or more sources of irrigation fluid, suction, or both may be controlled by one or more control modules.

The one or more control modules may function to control the amount of fluid, suction, or both applied to a predetermined area, an area of interest, the endoscope, or a combination thereof. The one or more control modules may control the order of application of fluid, suction, or both relative to each other. The one or more control modules may function to stop suction, stop irrigation fluid, apply irrigation fluid, apply suction, or a combination of both. The one or more control modules may include one or more pumps, one or more valves, one or more user interfaces, or a combination thereof. The one or more control modules may include any of the pumps discussed herein and based upon feedback from the user interface may control the pump to perform the selected parameter. The one or more control modules may control one or more valves. The one or more control modules may prevent application of suction and irrigation fluid at the same time. The one or more control modules may control one or more valves so that suction, irrigation fluid, or both may be applied in series, in parallel, simultaneously, one at a time, or a combination thereof. The one or more control modules may be physically connected, electrically connected, or both to one or more suction lines, one or more irrigation lines, one or more valves, or a combination thereof.

The one or more control modules may be located at an elevated position so that a user may adjust the control module, activate the control module, or both using hand controls (e.g., a console). The one or more control modules may be located on a floor position so that the user may adjust the control module using one or both feet. The one or more control modules may be part of a foot switch, a foot activation device, a foot petal, or a combination thereof. Functionality of the control module may be separated between a foot petal (e.g., a foot console) and an elevated portion of the control module (e.g., a hand console) and one or both may be used to control an actuation cycle. The control module may be connected to one or more pumps.

The pump may function to circulate irrigation fluid, move irrigation fluid through one or more lines, move fluid through a sheath, move irrigation fluid to a resilient reservoir, or a combination thereof. The pump may function to create a negative pressure (e.g., suction or vacuum). Preferably, the pump moves a constant amount of fluid upon being activated, a constant amount of fluid may be varied from application to application, or both. More preferably, the pump is a peristaltic pump. The pump may supply irrigation fluid through the irrigation lines.

The one or more irrigation lines may function to connect the sheath to an irrigation source, a resilient reservoir, one or more valves, a pump or a combination thereof. The one or more irrigation lines may extend through a control module. The irrigation line may be flexible, movable, or both. The irrigation lines may be compressible. One or more and preferably a plurality of irrigation lines may extend between an irrigation source, a resilient reservoir, a control module, and the sheath. The one or more irrigation lines may connect to one or more entrance ports, one or more exit ports, or both of a sheath, a resilient reservoir, an irrigation source, a pump, or a combination thereof. The irrigation lines may be made of any material that is compatible with the irrigation fluid, a patient, use in a surgical procedure, or a combination thereof. The irrigation lines may connect the sheath to an irrigation source, a suction source, or both (i.e., suction may be applied through the irrigation line).

The suction source may function to remove fluid, debris, opaque fluids, unwanted material, or a combination thereof from a point of interest, from a distal end of the sheath, a distal end of the endoscope, or a combination thereof. The suction source may function to perform a drying function, remove fluid spots, or both. The suction source may be a pump, reversal of a motor, a common suction source, a hospital suction source, or a combination thereof. The suction source may apply a sufficient amount of vacuum to remove a predetermined amount of fluid in a predetermined amount of time. For example, the suction source may apply suction so that 10 ml of fluid may be removed in 1 to 2 seconds. The suction source may apply suction for a predetermined amount of time. The predetermined amount of time may be about 2, 3, 4, 5, 6, 7, 8, 9, 10, 15, or 20 second or more. The suction source may provide suction immediately after the irrigation source ceases to provide irrigation fluid, after a delay, or both. The suction source may apply a continuous suction, intermittent suction, or both.

The suction line may function to connect the sheath to the suction source so that suction may be pulled through the sheath. The suction line may function to connect the sheath to a suction source. The suction line may assist is moving fluids, removing fluids, removing debris, removing opaque fluids, removing particles, or a combination thereof. The suction line may assist in creating a vacuum at a distal tip of the endoscope, the sheath, or both. The suction line and the irrigation line may be the same line. The suction line and the irrigation line may be connected to a common line. The suction line and the irrigation line may be connected by one or more fittings, one or more valves, or both. The suction line, the irrigation line, or both may be connected to a delivery line, a common line, or both.

The delivery line may function to deliver fluids to a sheath. The delivery line may function to deliver suction to the sheath. The delivery line and the common line are preferably the same line. The teachings herein for the common line are incorporated by reference here for the delivery line. The delivery line, common line, or both may be used during an application cycle to clean an endoscope.

The endoscope may function to provide an image to a surgeon, a doctor, a nurse, any other person who desires visual access to a remote location. The endoscope may be used for non-invasive surgery. The endoscope may be used for orthoscopic surgery. The endoscope may be inserted in a cut in tissue. The endoscope may have a generally circular cross-section. The endoscope may have a tubular section that is generally cylindrical (i.e., internal portion). Preferably, the endoscope has at least an end that is cylindrical. The endoscope may have a tubular section extending to the distal end and a handpiece connected to the tube and extending to the proximal end. The endoscope may have a cylindrical distal end. The body of the endoscope and the distal end of the endoscope may be different shapes. The endoscope may include one or more image sensors in a distal end region (i.e., internal portion). The one or more image sensors may be located in an external portion of the endoscope and fiber optics connected to the image sensor may transmit a signal through the internal portion to the external portion. The endoscope may include two or more image sensors. The endoscope may include an image sensor at the most distal point of the endoscope. The endoscope may include an image sensor that is located on an angle. The angle of the image sensor, viewing face, lens, or a combination thereof may be about 0°, 20°, 30°, 45°, 60°, 70°, or a combination thereof. The image sensor may provide black and white images, color images, thermal images, or a combination thereof. Preferably, the image sensor, imaging device, or both are located substantially at the distal end. The angle of the image sensor, the viewing face, lens, or a combination thereof may dictate the angle, shape, viewing cone, or a combination thereof of the endoscope.

The viewing cone may be an area of visibility of the endoscope. The viewing cone may be variable, adjustable, or both. The angle of the viewing cone may be movable. The angle of the viewing cone may be predetermined based upon the type of endoscope selected. The angle of the viewing cone may not be affected by the flow director, lumen, sheath, or a combination thereof. The location of the endoscope within the sheath may vary based upon the angle of the viewing cone. For example, the shape of the sheath may offset the endoscope to one side more or less based upon the angle of the viewing cone so that the endoscope sheath does not interfere with the imaging of the endoscope. The viewing cone may extend outward from the distal end of the endoscope in a cone shape.

The distal end of the endoscope may function to be inserted into a patient so that a feature of interest may be viewed through minimally invasive means. The distal end of the endoscope may be the leading portion of the endoscope (i.e., the first portion that enters a patient). The distal end of the endoscope may be on an opposing end of the endoscope as a proximal end. The proximal end may function to be gripped by a user. The proximal end may function to provide controls to a user. The proximal end may provide an interface for connecting other functional components such as an imaging device (e.g., a camera). The proximal end may function to provide power, sensing, suction, fluid, control, a connection point to outside devices, or a combination thereof to the distal end of the endoscope. The proximal end may be retained out of the patient and the distal end may be inserted in the patent. The proximal end may include one or more visual ports.

The visual port may function to provide a viewing window for a user. The visual port may function to allow a user to observe a feature of interest. The visual port may function to provide an output so that an image is displayed on a monitor. The visual port may provide visual access through the endoscope to a user. The visual port may be an optical window at the proximal end that provides visual access to a viewing lens at the distal end.

The viewing lens may function to provide a window that an image sensor views through. The viewing lens may function to protect an image sensor (e.g., a camera). The viewing lens may be a cover over an image sensor. The viewing lens may be a viewing face of the endoscope and vice versa. The viewing face may be a surface of the endoscope that an image is generated through. The viewing lens may be a portion of the endoscope extended into a location of interest and may be inhibited from allowing a clear image to be formed (i.e., at the distal end) (e.g., by opaque fluids such as blood). The viewing lens may be illuminated by light that extends from a light post in the endoscope.

The light post may function to provide light into the endoscope. The light post may direct light into the endoscope and out of the tube of the endoscope so that a feature of interest is illuminated. The light post may provide light so that a user can see features of interests that are located in low light conditions. The one or more light posts may provide light through the endoscope, so that the visual port may be used for observing a feature of interest at a distal end of the endoscope. The light post may be located proximate to one or more ports.

The one or more ports may function to provide irrigation fluid, suction, or both to the endoscope, the endoscope sheath (i.e., sheath), or both. The one or more ports may connect to a common line, a delivery line, a suction line, an irrigation line, or a combination thereof. The one or more ports may provide access through the endoscope, to the endoscope sheath, to the distal end, or a combination hereof. A single port may be present that provides both irrigation fluid and suction to a distal end of the sheath, the endoscope, or both.

The sheath may function to provide one or more conduits, lumen, channels, or a combination thereof for a fluid, suction, a functional device (e.g., a cutting tool, cauterizing tool, or both), or a combination thereof to extend out of a distal end region of the sheath. The sheath may function to form all or a portion of a conduit, channel, lumen, or a combination thereof for fluid, suction, a functional device, or a combination thereof to extend out of a distal end region of the sheath. The sheath may function to provide cleaning, washing, or both of an endoscope. The sheath may provide a conduit, channel, a lumen, or a combination thereof that extends from a proximal end to a distal end. The sheath may include one or more lumen, create one or more lumen, or both. The sheath may include one or more parts that when connected together create a conduit that provides irrigation fluid, suction, a functional device, or a combination thereof to a distal end of the endoscope. The sheath may substantially mirror the shape of the endoscope. Thus, for example, if the endoscope has a circular cross-section then then sheath has a circular cross section. The sheath may function as an endoscope cleaner. The sheath may have a distal end and a proximal end with a longitudinal axis that extends therebetween.

The distal end of the sheath may function to direct irrigation fluid, suction, or both across the viewing lens, the distal end, or both of the endoscope. The distal end of the sheath may include a distal end region that may include one or more of the features discussed herein. The distal end region may include one or more positioning devices, a plurality of positioning devices, one or more drip retention features, a plurality of drip retention features, one or more stoppers, a plurality of stoppers, engraving, or a combination thereof. The distal end region may be located proximate to the distal end but extend towards the proximal end away from the distal end. The distal end region may be located entirely on one side of a mid-point of the endoscope sheath. The distal end region may have a length that is about 40 percent or less, 35 percent or less, 30 percent or less, 10 percent or more, 15 percent or more, 20 percent or more, or even about 25 percent or more of the total length of the endoscope sheath when measured from the distal end.

The proximal end of the sheath may function to create a connection with the endoscope. The proximal end may align the sheath relative to the endoscope. The proximal end of the sheath may axially align the sheath relative to the endoscope, radially align the sheath relative to the endoscope, axially align the distal ends of the sheath and the endoscope, the sheath axially relative to a light post of the endoscope, the sheath rotationally relative to a light post of the endoscope, or a combination thereof. The proximal end may include a distal end region that may include one or more of the features discussed herein. The proximal end region may include one or more positioning devices, a plurality of positioning devices, one or more drip retention features, a plurality of drip retention features, one or more stoppers, a plurality of stoppers, engraving, or a combination thereof. The proximal end may receive all or a portion of the endoscope. The proximal end may contact a shoulder of the endoscope. A longitudinal axis may extend between the proximal end and the distal end of the sheath. The longitudinal axis may extend through a through hole, channel, lumen, tube, or a combination thereof that extends the length of the sheath. The endoscope may extend within the sheath along the longitudinal axis. The longitudinal axis may extend from a connection point between the endoscope and the sheath and through a tube of the sheath (e.g., a length of the tube of the sheath). The tube may define one or more through holes along the length of the tube.

The tube may function to receive the imaging device of the endoscope. The tube and the sheath may be configured to receive all or a portion of an endoscope. The tube and sheath may be configured to provide a conduit for communicating fluid between the proximal end of the endoscope sheath and the distal end of the endoscope sheath when the endoscope is inserted inside the sheath (or tube). The tube may be the main body portion of the endoscope sheath and when combined with one or more of the other features discussed herein form an endoscope sheath. The tube may have one or more positioning devices for positioning the endoscope within the sheath. The tube may include one or more positioning devices for creating a fluid path, a suction path, or both though the tube. The tube may be located at the distal end of the endoscope. The tube may be generally the same size and shape as the endoscope. For example, if the endoscope has a generally circular cross-section then the tube may have a generally circular cross-section. Preferably, the tube has at least a generally circular cross-section at the distal end.

A through hole may extend from a proximal end to a distal end of the tube (e.g., endoscope sheath). A through hole may be sufficiently large so that the endoscope and fluid (e.g., irrigation fluid, suction, or both) may pass from the distal end to the proximal end of the sheath. The tube may include one or more through holes in the sheath. The through hole in the tube may open directly to a point of interest, an internal location of a patient, or both. The through hole may include one or more of the features discussed herein.

The one or more positioning devices may function to position an endoscope within a sheath. The one or more positioning devices may function to axially align, radially align, longitudinally align, laterally align, or a combination thereof the endoscope within a sheath. The one or more positioning devices may extend along a portion of the length or the full length of the sheath, the tube of the sheath, or both (e.g., a surface of the tube). The one or more positioning devices may be located continuously between the distal end and proximal end of the sheath, periodically be located between the distal end and the proximal end of the sheath, or a combination of both. The one or more positioning devices may be spaced apart. The one or more positioning devices may be circumferentially spaced apart, longitudinally spaced part, laterally spaced apart, coplanar, non-coplanar, or a combination thereof. The one or more positioning devices may be in a line such that each of the positioning devices are coplanar and perpendicular to the longitudinal axis. The one or more positioning devices may be staggered and coplanar (e.g., circumferentially spaced apart and longitudinally spaced apart). The one or more positioning devices may be staggered and non-coplanar. The positioning devices may only be located in the distal end region, proximate to the distal end region, on the distal end side of the sheath, or a combination thereof. The positioning devices may be positioned in groups and/or sets. The positioning devices may create a space between the tube and the endoscope so that irrigation fluid, suction, or both can travel through the endoscope sheath. The positioning devices may form a space that is equal all of the way around the endoscope (i.e., 360 degrees). The positioning devices may create a gap between the drip retention features and the endoscope. For example, the height of the positioning devices may be greater than the height of the drip retention features. The positioning devices may offset the endoscope with thin the endoscope sheath so that a larger space is located on one side than an opposing side (e.g., eccentrically positioned). The positioning devices may be evenly spaced apart within the endoscope sheath (e.g., if there are three then they are all 120 degrees apart). The positioning devices may be unevenly spaced apart within the endoscope sheath (e.g., one may be located at 12 o'clock, one at 4 o'clock and one at 7 o'clock). The positioning devices may maintain the endoscope a distance from the end of the sheath. The distance may be a sufficient distance so that the irrigation fluids are moved across the lens, imaging device, or both by surface tension. The distance between the distal end of the sheath and the distal end of the endoscope may be a distance so that surface tension moves an irrigation fluid across the lens, the imaging device, or both. For example, surface tension may cause the irrigation fluid to wrap around the imaging device, lens, or both of the endoscope so that the endoscope is cleaned. The distance between the distal end of the endoscope and the distal end of the sheath may be about 1 mm or more, about 2 mm or more, or about 3 mm or more. The distance between the distal end of the endoscope and the distal end of the sheath may be about 15 mm or less, about 12 mm or less, or about 10 mm or less. The surface tension may maintain fluid in contact with the lens, the imaging device, or both so that the lens, the imaging device, or both are washed, cleaned, or both. The one or more positioning devices may both axially align the endoscope and position the endoscope within the sheath, the tube, or both.

The one or more positioning devices may align the endoscope within the tube, the sheath, or both. The one or more and preferably a plurality of positioning devices may create an annular gap around an endoscope. The annular gap may be uniform around the endoscope. The annular gap may vary in distance between the outer wall of the endoscope and the inner wall of the sheath. The one or more and preferably a plurality of positioning devices may move the endoscope into contact with a wall of a sheath, a tube, or both so that a gap is only created around a portion of the endoscope, a fluid is prevented from extending between a contact location between the endoscope and sheath, or both. An offset gap may be created so that a center of the sheath and a center of the endoscope are offset, eccentric, shifted relative to each other, or a combination thereof. For example, the endoscope may be shifted all the way to one wall so that a gap is only located on one side of the endoscope. The one or more positioning devices may function to be an axial stop. The one or more positioning devices may move the endoscope into contact with a surface (e.g., the tube, the sheath, or both) so that a fluid barrier is created.

The positioning devices may be an integral part, a unitary part, a non-unitary part, or a combination thereof of the sheath. The positioning devices may be added to the sheath, the tube, or both (i.e., non-unitary). The positioning devices may be a non-welded piece, non-soldered piece, or both that is added to the sheath, the tube, or both. The positioning devices may be an added piece of material that is connected to the sheath, the tube, or both. The positioning devices may be added without heating the positioning devices, the tube, the sheath, or a combination thereof (i.e., liquefying material or adding molten material). The positioning devices may be connected to the sheath, the tube of the sheath, or both by one or more fasteners. The positioning devices may be connected to the tube, the sheath, or both by an adhesive, a threaded connection, a rivet like connection, a friction fit, a mating member that extends through the tube and/or sheath, or a combination thereof. The positioning devices may form a connection so that the positioning devices extend out from an inside wall of the tube and/or sheath and form a substantially flush connection with an outside wall of the tube.

The one or more positioning devices may be a formed part of the sheath, the tube, or both such that no additional material is added (i.e., unitary). The one or more positioning devices may be a portion that is dented, formed, crushed, pressed, molded, or a combination thereof. The one or more positioning devices may be created by cutting a portion of the sheath, the tube, or both and repositioning the piece of cut material (e.g., a crimp). The sheath may be formed so that the sheath includes one or more positioning devices. The sheath may include a plurality of positioning devices. The positioning devices may be located on an inner wall, an outer wall, be part of the wall, extend through the wall, or a combination thereof of the sheath and/or tube. Preferably, the sheath includes two or more positioning devices.

The two or more positioning devices may be equally spaced apart. For example, if there are two positioning devices the devices may be 180 degrees apart and there are three positioning devices the positioning devices may be 120 degrees apart. The two or more positioning devices may be spaced apart about 15 degrees or more, about 30 degrees, or more, about 45 degrees or more, about 60 degrees or more, about 90 degrees or more, about 120 degrees or more, or even about 150 degrees or more apart. The two or more positioning devices may be located about 180 degrees or less or about 160 degrees or less apart.

The positioning devices may be a circular portion, a circular segment, a dimple, a pin, a crimp, an end stop (i.e., a stopper), a tangent portion, a tangent segment (or line), an oblique portion, an oblique segment (or line), or a combination thereof and the teachings as to the positioning devices are incorporated by reference herein for each of the various types of positioning devices from the teachings herein including U.S. Patent Application Publication No. 2015/0087907 and paragraphs 0071-0083 and FIGS. 4A-12C. A preferred positioning device is a dimple.

The one or more dimples may function to position an endoscope within a sheath, a tube of the sheath, or both. The one or more dimples may function to axially position the endoscope within the sheath (e.g., form a radial stop). The one or more dimples may function as a distal end stop, a locator, an axial locator, a cross-sectional locator (e.g., shift the sheath within the cross-section of the sheath), or a combination thereof. For example, the one or more dimples may be used to create an annular gap, an offset gap, or both. The one or more dimples may contact a point of the endoscope along the length of the endoscope. The one or more dimples may function to position the endoscope within the sheath so that a conduit, channel, lumen, space, or a combination thereof is created along all or a portion of the longitudinal axis of the endoscope, sheath, or both. The one or more dimples may create a space, a conduit, a lumen, a channel, or a combination thereof between a wall of the sheath and the endoscope. The one or more dimples may be a portion of the wall of the sheath that extends inward (e.g., towards a center of the sheath). The one or more dimples may be generally round, square, oval, triangular, rounded, have a flat surface, have a rounded surface, be hemispherical, or a combination thereof. The one or more dimples may be an indentation and/or deformation in the side of the sheath, the tube, or both without adding material, without removing material, without relocating material, or a combination thereof. The one or more dimples may be located on opposing sides of the tube. The one or more dimples may be radially spaced apart, axially spaced apart, longitudinally spaced apart, or a combination thereof. The one or more dimples may be located along the length. For example, the tube may include dimples that are spaced apart from the proximal end to the distal end so that the endoscope and sheath are fully supported relative to each other along their respective lengths. If more than one dimple is present the dimples may be located adjacent, in the same plane, in a line, be axially spaced apart, radially spaced apart, coplanar, non-coplanar, or a combination thereof. When more than one dimple is present the dimples may be in a straight line relative to the longitudinal axis, perpendicular to the longitudinal axis, at an angle relative to the longitudinal axis, or a combination thereof. When more than one dimple is present the dimples may be separated by an angle of about 180 degrees or less, about 150 degrees or less, about 120 degrees or less, about 90 degrees or less, or even about 60 degrees or less. The two or more dimples may be separated by an angle of about 15 degrees or more, about 30 degrees or more, or even about 45 degrees or more. The sheath may include about 2 or more dimples, 3 or more dimples, 4 or more dimples, 5 or more dimples, or even about 6 or more dimples. Two or more dimples may be located generally within the same plane (e.g., around an inner circumference of the tube at an equal distance from the distal end) and radially spaced apart so that the dimples offset the endoscope within the sheath (e.g., the center of the endoscope and the center of the sheath are not in line). The one or more dimples may be located on the same side of the sheath as the port, opposite side of the sheath as the port, at an angle relative to the port, or a combination thereof. The one or more dimples may be used in conjunction with one or more tangent segments, one or more oblique segments, one or more pins, one or more crimps, one or more end stops, or a combination thereof. The one or more dimples may locate the endoscope relative to one or more drip retention features.

The one or more drip retention features may function to retain a volume of liquid by capillary action. The one or more drip retention features may function to substantially prevent and/or substantially eliminate liquids from forming on a lens, an imaging device, or both. The one or more drip retention features may eliminate free fluids from being present on the lens, the imaging device, or both. The one or more drip retention features may function to pull back liquids when the irrigation fluid, suction, or both are turned off. The one or more drip retention features may prevent fluid form being located distal of the distal end of the endoscope. The one or more drip retention features may prevent fluid from dripping across the lens and/or imaging device after cleaning. Each sheath may include one or more drip retention features. Each sheath may include a plurality of drip retention features. The drip retention features may be used with any of the endoscopes discussed herein. Preferably, the drip retention features are located with the 0 degree endoscopes (i.e., an endoscope with a substantially flat face). The one or more drip retention features may have a shape that when viewed in a cross section is semi-circular, crescent moon shaped, square, has an apex, has multiple apexes, is “M” shaped, is “W” shaped, includes a channel, a concave portion, raised surfaces, or a combination thereof. The one or more drip retention features may include one or more recesses, one or more voids, one or more channels, one or more depressions, one or more concave portions, or a combination thereof. The one or more drip retention features may be generally arcuate. The one or more drip retention features may have a shape that when the drip retention features contact the endoscope there is point contact in the cross-section and line contact along the length (i.e., in three dimensional). The one or more drip retention features may have one or more locations of contact, two or more locations of contact, or even three or more locations of contact with an endoscope. The drip retention features may be free of contact with the endoscope along all or a portion of a length of the drip retention features. The drip retention features may be located at any location within the inner circumference of the sheath. Preferably, the drip retention features are located in the bottom half (i.e., the half between 3 o'clock and 9 o'clock). More preferably, the drip retention features are located at the bottom (i.e., 6 o'clock). The drip retention features may wrap around a portion of the inner circumference of the tube. The drip retention features may extend substantially parallel with the longitudinal axis. The drip retention features may extend through the tube linearly, helically, canted, or a combination thereof. The drip retention features may be located so that a distal end of the drip retention features are at the distal most end of the sheath. Preferably, the drip retention features are located proximal of the distal end of the endoscope, the endoscope sheath, or both. The drip retention features may be located about 5 percent or more, 8 percent or more, about 10 percent or more, or even about 15 percent or more of the length of the sheath from the distal end. The drip retention features may be located about 50 percent or less, 40 percent or less, about 25 percent or less, or even about 20 percent or less of the length of the sheath from the distal end. For example, if the sheath has a length of about 200 mm and the drip is located about 10 percent of the total length from the distal end the drip retention features will be 20 mm from the distal end. The drip retention features may be located about 5 mm or more, about 8 mm or more, about 10 mm or more, or preferably about 12 mm or more from the distal end of the endoscope sheath. The drip retention features may be located about 40 mm or less, about 30 mm or less, about 20 mm or less, or even about 15 mm or less from the distal end of the endoscope sheath. The distal most end of the drip retention features may be in line with, distal of, proximal of, or a combination thereof the positioning devices.

The one or more drip retention features have a length, a width, and a height. The length, width, height, gap, or a combination thereof of the drip retention features may define a volume that functions to hold free fluids, free liquids, or both (e.g., free fluid may be any fluid that is between the endoscope and the endoscope sheath that may move to inhibit vision) so that drips are prevented across the lens, the imaging device, or both (e.g., the length, width, and height are selected to create a predetermined volume). Stated another way the length, height, width, and gap create a volume that may be filled with free fluid. The length of the drip retention features may be sufficiently long so that drips are prevented after a cleaning cycle. The length of the drip retention features may vary depending upon the height and/or width of the drip retention features. The length of the drip retention features may be the same length as the sheath. Preferably, the length of the drip retention features are less than the length of the sheath. The drip retention features may have a length that is about 75 percent or less, 60 percent or less, or even about 50 percent or less than the length of the sheath. The drip retention features may have a length that is about 10 percent or more, about 25 percent or more, or even about 40 percent or more of the length of the sheath. The length of the drip retention features may be about 5 mm or more, about 10 mm or more, about 12 mm or more (i.e., about 16 mm). The length of the drip retention features may be about 5 cm or less, about 3 cm or less, about 25 mm or less, or about 20 mm or less. The length of the drip retention features may have a ratio relative to the width. The ratio of the length to width of the drip retention features may be about 3:1 or more, about 5:1 or more, and preferably about 7:1 or more, more preferably about 10:1 or more, and most preferably about 12:1 or more. The ratio of the length to width of the drip retention features may be about 75:1 or less, about 50:1 or less, about 30:1 or less, or about 20:1 or less.

The width of the drip retention features may function to be sufficiently large so that surface tension of the free fluid pulls the free fluid into the volume creating a space for more free fluid to be pooled. The width of the drip retention features may be sufficiently large so that free fluid is removed from the lens, the imaging device, or both. The width of the drip retention features may be sufficiently large so that no free fluid is located at the distal end of the endoscope after a cleaning cycle. The width of the drip retention features may be substantially equal along the length of the sheath. The width of the drip retention features may taper along the length of the sheath. The width of the drip retention features may vary along the length. The drip retention features may have its largest width at the distal end, the proximal end, or some location therebetween. The drip retention features may be bowtie shaped (i.e., have a smaller center and larger ends). The one or more drip retention features alone or in combination may span about 1 percent or more, about 3 percent or more, about 5 percent or more, or even about 10 percent or more of the circumference of the sheath. For example, if the width is about 10 percent then the drip retention features will span about 36 degrees of the inner circumference of the sheath. The one or more drip retention features along or in combination may span about 80 percent or less, preferably about 50 percent or less, or more preferably about 30 percent or less of the circumference of the sheath. The drip retention features may have a width of about 0.2 mm or more, about 0.5 mm or more, about 0.8 mm or more, about 1.3 mm or more. The drip retention features may have a width of about 5 mm or less, about 3 mm or less, or about 2 mm or less. The drip retention features may have a ratio of width to height. The ratio of width to height may be about 1:1, about 1:1.2 or more, about 1:1.4 or more, or about 1:1.5 or more. The ratio of width to height may be about 1.5:1 or less, about 1.4:1 or less, or about 1.2:1 or less.

The height of the drip retention features may function to contact free fluid and to create a volume or space into which surface tension draws the fluid into a cavity. The height of the drip retention features may be sufficiently large so that the drip retention features contact free fluid, prevents fluid from being maintained on the lens, the imaging device, or both. The height of the drip retention features may be measured at a highest point, each peak, a mid-point, or a combination thereof. Preferably, the drip retention features have at least one apex and the height is measured at the at least one apex. Although if more than one apex is present, each apex will have its own height. The height of the drip retention features may be about 0.3 mm or more, about 0.5 mm or more, about 0.8 mm or more, about 1.0 mm or more, or about 1.2 mm or more, The height of the drip retention features may be about 3 mm or less, about 2 mm or less, or about 1.5 mm or less. The drip retention features may have a ratio of the height of the drip retention features to the height of the positioning feature of about 1:1, about 1.1:1 or more, or about 1.2:1 or more. The ratio of the height of the drip retention features to the height of the drip retention features are preferably about 1:1 or less, about 0.95:1 or less, about 0.9:1 or less, or about 0.8:1 or less. The drip retention features may have a height that is less than the height of the positioning feature so that the positioning feature contacts the endoscope and the drip retention feature is free of contact with endoscope (e.g., the drip retention features have a height of about 1.35 mm and the positioning feature has a height of about 1.4 mm). The drip retention features may have a length to height ratio of about 3:1 or more, preferably about 5:1 or more, and more preferably about 7:1 or more. The ratio of length to height of the drip retention features may be about 20:1 or less, about 15:1 or less, or about 12:1 or less. The height of the drip retention features may vary along the length of the drip retention features. The height of the drip retention features may be at a maximum at the distal end, the proximal end, or a location therebetween. The height of the drip retention features may be sinusoidal. The one or more drip retention features may have a shape so that a volume is formed between the drip retention features and the endoscope.

The one or more drip retention features may have a volume that is filled with fluid (e.g., a liquid and preferably saline) after each cleaning cycle is completed, after the irrigation fluid is stopped, after suction is stopped, or a combination thereof. The one or more drip retention features may function to collect, retain, attract, or a combination thereof all of the free fluids of the system, preferably the free liquids, and more preferably, the free saline or cleaning fluids. The one or more drip retention features may have a sufficient volume so that when the irrigation fluid, suction, or both are stopped the remaining fluid is all drawn to the drip retention features. The drip retention features may have a volume of about 5 ml or more, about 10 ml or more, about 15 ml or more, about 20 ml or more. The drip retention features may have a volume of about 100 ml or less, about 75 ml or less, and about 50 ml or less. The volume the drip retention features can retain may be directly related to the length, width, height, gap, or a combination thereof of the drip retention features. The volume the drip retention features can retain may be related to the size of the gap between the drip retention features and the endoscope. The drip retention features may be smooth. The drip retention features may be free of surface treatments. The drip retention features may include a surface treatment. The surface treatment may function to increase surface area, surface tension, or both. The surface treatment may be engravings, pores, a microstructure, a roughed surface, a sprayed on treatment, painted on treatment, an etching, a treatment with micro particles that create a raised surface, or a combination thereof. The drip retention features may be made of the same material and in the same manner as the tube, the sheath, or both. The drip retention features may be made of metal, a biocompatible material, plastic, rubber, or a combination thereof. The volume of fluid that may be retained by the drip retention features may be related to the length, width, height, surface treatment, a gap, or a combination thereof.

The gap between the drip retention features and the endoscope may function to create a volume that retains free fluids (e.g., saline, cleaning fluid, or both). The gap between the drip retention feature sand the endoscope may be sufficiently small so that the drip retention features contact free fluid, prevents free fluid from migrating to the distal end of the lens, the imaging device, or both and restricting a user's vision. The gap may be sufficiently large so that the drip retention features retains all of the free fluid and restricts free fluid from interfering with the lens, the imaging device, or both. The gap may not be present (i.e., 0 gap). If a gap is not present the drip retention features may contact the endoscope but the drip retention features may not position the endoscope within the sheath. Preferably, a least a slight gap is present between the endoscope and the drip retention features (e.g., 5 microns or more, 10 microns or more, or even 1 mm or more). The gap may be difference between the height of the drip retention features and the positioning feature. The gap may be a distance between the drip retention features and endoscope of about 0.1 mm or more, about 0.2 mm or more, about 0.3 mm or more, about 0.5 mm or more, about 1 mm or more, or about 2 mm or more. The gap may be a distance between the drip retention features and endoscope of about 5 cm or less, about 4 cm or less, about 3 cm or less, about 2 cm or less, about 1 cm or less, or about 5 mm or less. The gap may vary along the length so that free fluid is moved from a location with a smaller gap to a location with a larger gap or vice versa. For example, at the distal end the gap may be about 3 mm and at the proximal end may be about 5 mm. The gap may be smallest at the distal end, towards the proximal end, or a location therebetween. The gap may be formed by the positioning devices having a length that is greater than the length of the drip retention features. The gap may be formed by the endoscope being eccentrically located within the sheath. The gap may be located between some portions of the drip retention features and not present at other locations of the drip retention features. For example, if the drip retention features have a W shape, the three apexes may contact the endoscope and gaps may be located between the apexes for retaining free fluid. The drip retention features may be located proximate to a stopper so that the endoscope and the sheath are positioned so that the drip retention features may remove free fluids from the distal end.

The stopper may function to align a distal end of the endoscope within the sheath. The stopper may prevent the endoscope from extending out of the sheath. The stopper may be a positioning feature. The stopper may align the distal ends of the endoscope and the endoscope sheath within interfering with the lens, the imaging device or both.

The endoscope may include engravings, surface treatments, or both (hereinafter referred to as engravings). The engravings may function to align the endoscope with the endoscope sheath. The engravings may function to assist a user in aligning the endoscope and endoscope sheath. The engravings may be linear, circular, a geometric shape, or a combination thereof. The engravings may be located on a top, a bottom, a side, or a combination thereof. The engravings may be a pattern. The engravings may only be located on an outside or an inside of the endoscope sheath. The engravings may be a laser engraving. The engravings may span a perimeter of the sheath. The engravings may be continuous, intermittent, spaced apart, along a portion of the endoscope.

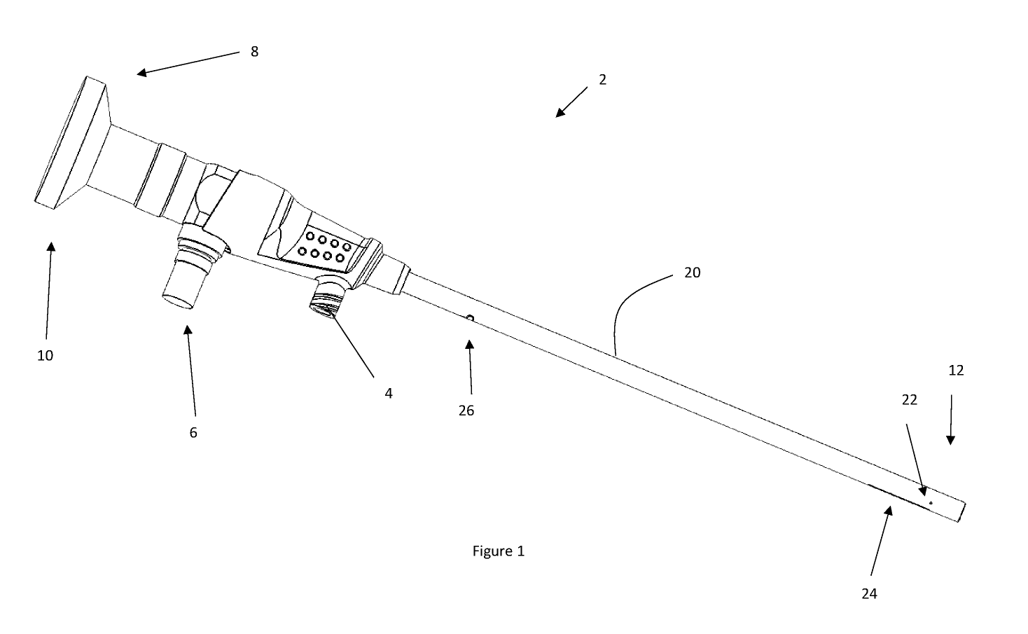

FIG. 1 illustrates a side perspective view of an endoscope 2. The endoscope 2 includes a proximal end 10 and a distal end 12. The endoscope 2 has a port 4 and a light post 6 at the proximal end 10 located near the visual port 8. The endoscope 2 has a portion that is covered by an endoscope sheath 20. The sheath 20 includes engraving 26 between the proximal end 10 and distal end 12. The endoscope sheath 20 includes positioning devices 22 and a drip retention feature 24 towards the distal end 12.

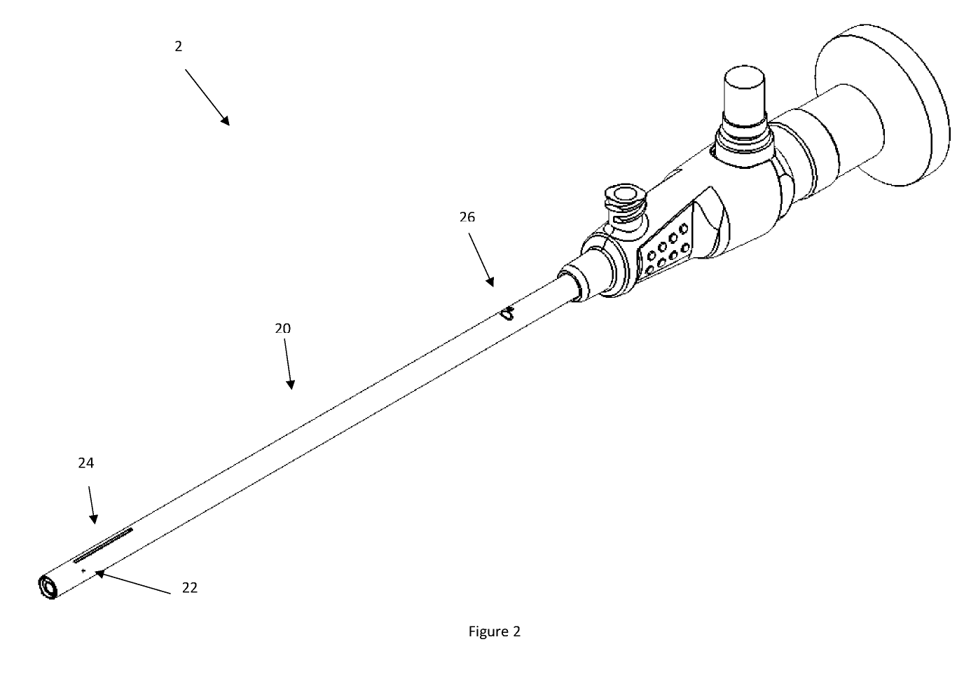

FIG. 2 illustrates perspective view of a bottom of an endoscope 2. An endoscope sheath 20 covers a portion of the endoscope 2. The endoscope sheath 20 includes engraving 26, positioning devices 22, and drip retention features 24.

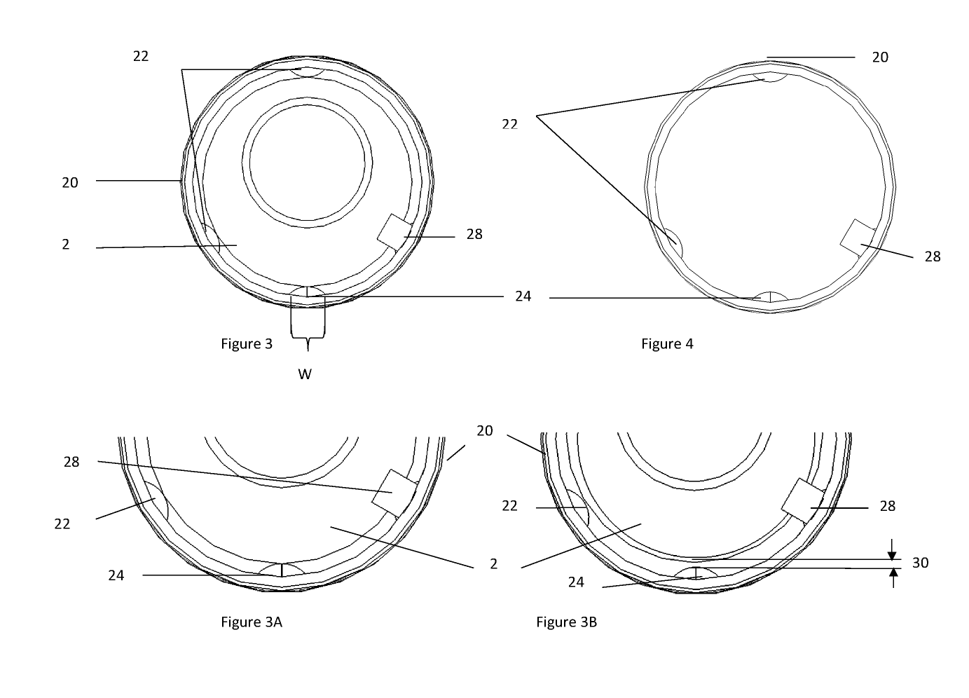

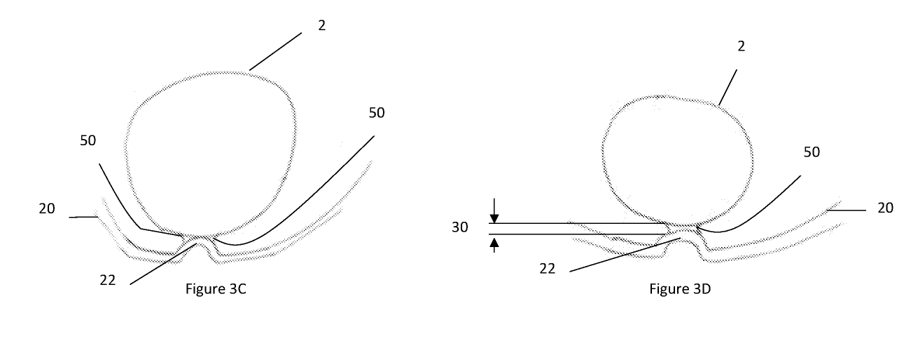

FIG. 3 illustrates a close-up of a distal end of an endoscope 2 located within an endoscope sheath 20. The endoscope sheath 20 includes a stopper 28 that aligns an end of the endoscope 2 and endoscope sheath 20. Positioning devices 22 assist in aligning the endoscope 2 within the endoscope sheath 20. A drip retention feature 24 as shown has a width (W) and is in communication with the endoscope 2 and prevents a fluid from being retained on or building up on a lens of the endoscope 2.

FIG. 3A illustrates a close-up view of the distal end of the endoscope 2 and the endoscope sheath 20. As shown the positioning devices 22 align the endoscope 2 so that the endoscope 2 is in contact with the drip retention feature 24. The stopper 28 stops the endoscope 2 so that the end of the endoscope 2 and the end of the endoscope sheath 20 are aligned.

FIG. 3B illustrates a close-up view of the distal end of the endoscope 2 and the endoscope sheath 20. As shown, the positioning devices 22 align the endoscope 2 so that a gap 30 is located between the endoscope 2 and the drip retention feature 24. The stopper 28 stops the endoscope 2 so that the end of the endoscope 2 and the end of the endoscope sheath 20 are aligned.

FIG. 3C illustrates a close-up view of the drip retention feature 22 of the endoscope sheath 20. The drip retention feature 22 is in contact with the endoscope 2 and fluid 50 is located in the volume on both sides of the drip retention feature 22.

FIG. 3D illustrates a close-up view of the drip retention feature 22 of the endoscope sheath 20. A gap 30 is located between the drip retention feature 22 and the endoscope 2. A single volume is formed in the gap 30 and the volume includes the fluid 50.

FIG. 4 illustrates a close-up view of an endoscope sheath 20. The endoscope sheath 20 as shown includes positioning devices 22 for centering the endoscope (not shown) within the endoscope sheath 20. A drip retention feature 24 is located at six o'clock. A stopper 28 is located at the end to longitudinally align the end of the endoscope (not shown) with the end of the endoscope sheath 20.

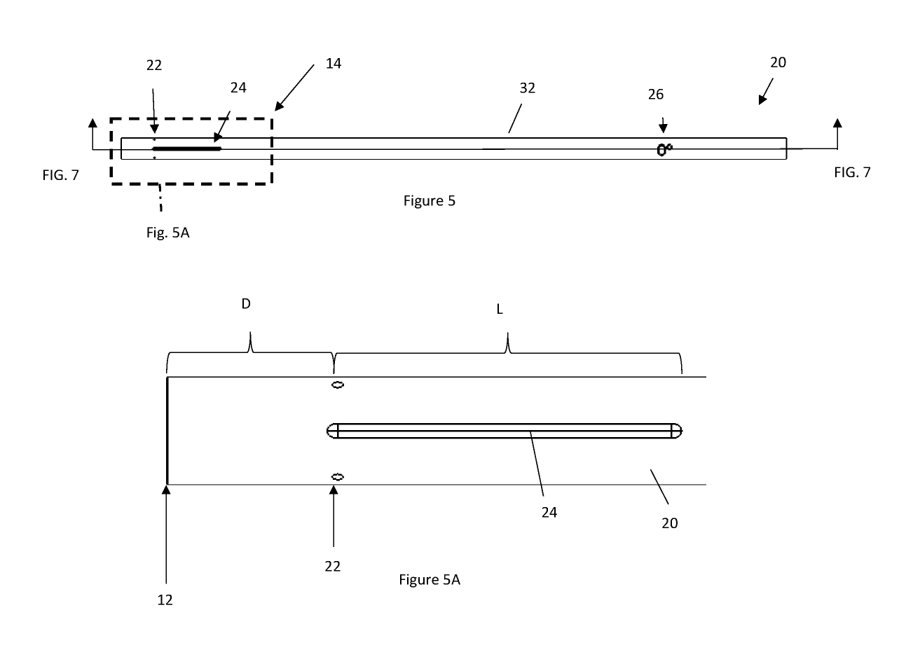

FIG. 5 illustrates a bottom plan view of an endoscope sheath 20. The endoscope sheath 20 includes a tube 32 and one end of the tube 30 includes engravings 26. A second opposing end includes positioning devices 22 and drip retention features 24 in a distal end region 14.

FIG. 5A illustrates a close-up view of a distal end 12 of an endoscope sheath 20. The distal end region at the distal end 12 includes positioning devices 22 and drip retention features 24. The positioning devices 22 and drip retention features 24 are located a distance (D) from the distal end 12. The drip retention features 24 have a length (L).

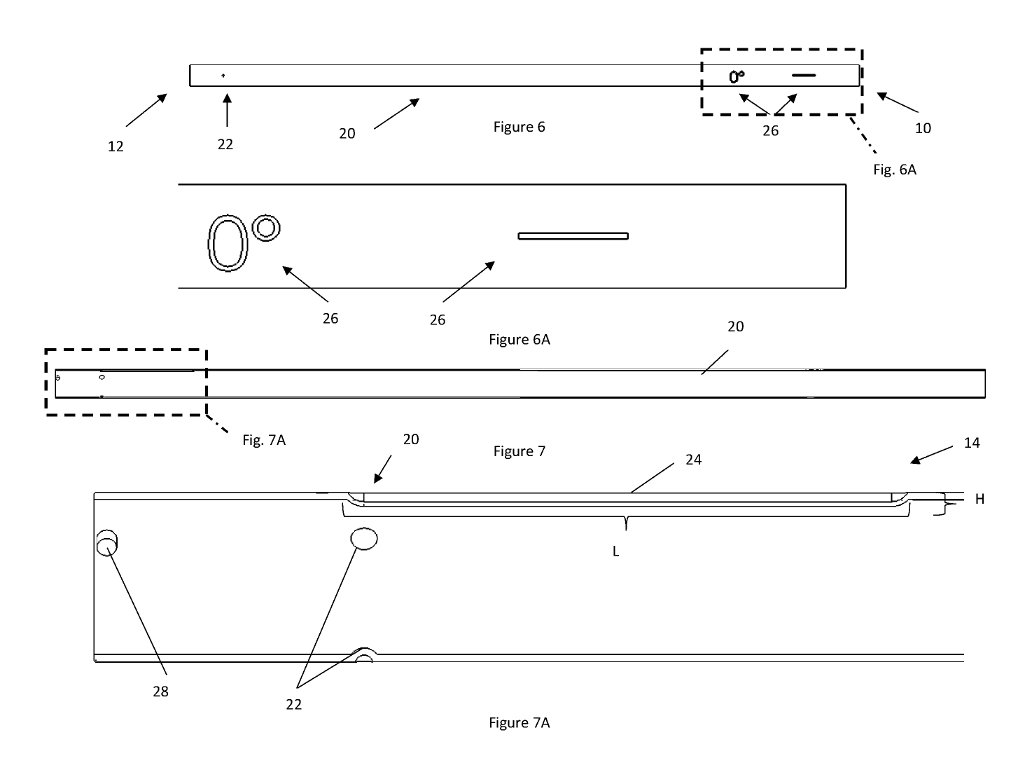

FIG. 6 illustrates a top view of the endoscope sheath 20 with positioning devices 22 located at the distal end 12 and engravings 26 located at the proximal end 10.

FIG. 6A illustrates a close-up view of the distal end of FIG. 6 with engravings 26 that are generally circular and engravings 26 that are linear.

FIG. 7 illustrates a cross-sectional view of the endoscope sheath 20 of FIG. 5 along lines 7-7.

FIG. 7A illustrates a close-up view of the distal end region 14 of FIG. 7. The distal end region 14 includes a stopper 28 at the most distal location of the endoscope sheath 20. The endoscope sheath 20 includes positioning devices 22 that are substantially aligned with the distal end of the drip retention feature 24. The drip retention feature 24 includes a length (L) and a height (H).

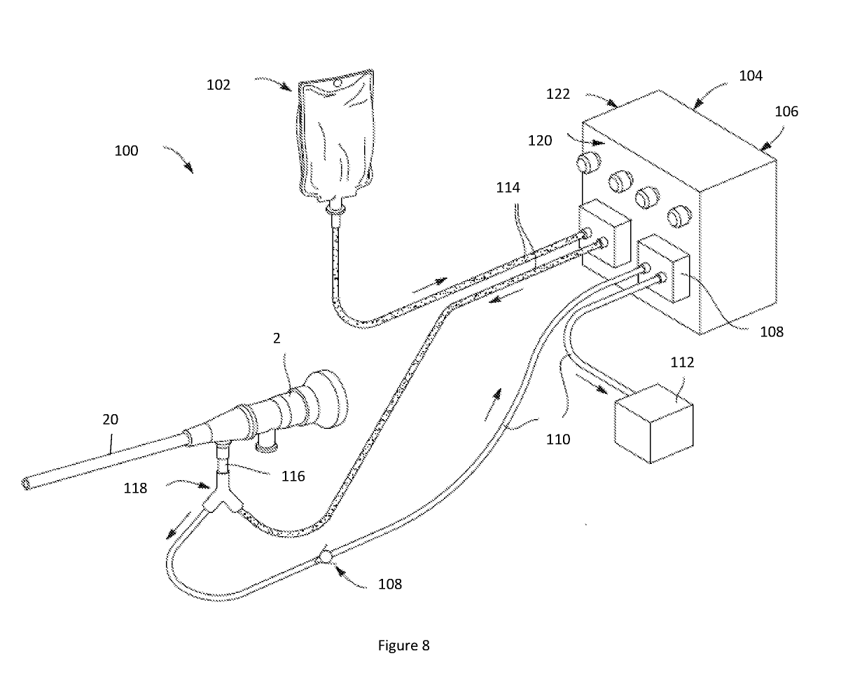

FIG. 8 illustrates a system 100 including the endoscope sheath 20 on the endoscope 2. The endoscope 2 and endoscope sheath 20 are connected to a control module 120 with a user interface 122. The control module includes a pump 104 and a power supply 106. An irrigation source 102 extends into the control module 120 through an irrigation line 114. The irrigation fluid is then pumped out of the control module 120 through an irrigation line 114 that extends into a common line 116. The common line 116 is connected to both the endoscope 2 and to the suction source 118 so that once the application of fluid is complete the suction removes excess fluid through the suction line 110. The suction line 110 includes a valve 108 so that suction is turned off during the application of fluid. The suction line 110 is connected to a suction source 112, which extends into the control module where a second valve 108 is located.

Any numerical values recited herein include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference for all purposes. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of the elements, ingredients, components or steps. By use of the term “may” herein, it is intended that any described attributes that “may” be included are optional.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

It is understood that the above description is intended to be illustrative and not restrictive. Many embodiments as well as many applications besides the examples provided will be apparent to those of skill in the art upon reading the above description. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.
2 Endoscope
4 Port
6 Light Post
8 Visual port
10 Proximal end
12 Distal end
14 Distal end region
20 Endoscope Sheath
22 Positioning Feature
24 Drip retention feature
26 Engraving
28 Stopper
30 Gap
32 Tube
50 Fluid
100 system
102 Irrigation source
104 Pump
106 Power supply
108 valve
110 Suction line
112 Suction source
114 Irrigation line
116 Common line
118 Common fitting
120 Control module
122 User interface
D Distance from Distal end
L Length of drip retention feature.
H Height of drip retention feature
W Width of drip retention feature

We claim:

1) An endoscope sheath comprising:
a. a proximal end,
b. a distal end having a distal end region,
c. a surface extending between and connecting the proximal end and the distal end,
d. a plurality of positioning devices located along the surface; and
e. one or more drip retention features;
wherein the sheath is configured to:
i. receive all or a portion of an endoscope and
ii. provide a conduit for communicating fluid between the proximal end of the endoscope sheath and the distal end of the endoscope sheath when the endoscope is inserted inside the sheath; and
wherein the plurality of positioning devices are located in a distal end region at the distal end of the endoscope sheath so that the endoscope is secured within the endoscope sheath; and
wherein the one or more drip retention features are configured to produce a region bounded by the endoscope sheath and the endoscope to retain a volume of liquid by capillary action.

2) The endoscope sheath of claim 1, wherein the one or more drip retention features are free of contact with the endoscope.

3) The endoscope sheath of claim 1, wherein a gap is located between the one or more drip retention features and the endoscope.

4) The endoscope sheath of claim 1, wherein the one or more drip retention features are in contact with the endoscope.

5) The endoscope sheath of claim 1, wherein the volume of the liquid is sufficiently large that any free liquid is drawn to the drip retention features and prevented from interfering with the endoscope.

6) The endoscope sheath of claim 1, wherein the one or more drip retention features have a length that is less than a length of the endoscope sheath.

7) The endoscope sheath claim 1, wherein the one or more drip retention features have a length to width ratio that is about 3:1 or more.

8) The endoscope sheath of claim 1, wherein a length of the one or more drip retention features are about 3 mm or more and about 5 cm or less.

9) The endoscope sheath of claim 1, wherein a width of the one or more drip retention features is about 0.2 mm or more, and about 5 mm or less.

10) The endoscope sheath of claim 1, wherein a width of the one or more drip retention features varies along a length of the one or more drip retention features.

11) The endoscope sheath of claim 1, wherein a height of the one or more drip retention features is about 0.3 mm or more.

12) The endoscope sheath of claim 1, wherein the one or more drip retention features have a length to height ratio that is about 3:1 or more.

13) The endoscope sheath of claim 1, wherein a height of the one or more drip retention features vary along a length of the one or more drip retention features.

14) The endoscope sheath of claim 1, wherein the one or more drip retention features extend linearly, helically, canted, or a combination thereof from the proximal end to the distal end.

15) The endoscope sheath of claim 1, wherein the drip retention feature includes: a surface treatment, free of a surface treatment, smooth, engravings, a semi-circular shape, a square shape, an apex, a point, an “M” shape, a “W” shape, a channel, recesses, voids, one or more through holes, one or more raised surfaces, or a combination thereof.

16) The endoscope sheath of claim 1, wherein the endoscope sheath includes an outside and at least a portion of the outside includes a pattern.

17) The endoscope sheath of claim 16, wherein the pattern is laser engraved on the outside of the endoscope sheath.

18) The endoscope sheath of claim 16, wherein the pattern spans around an entire perimeter along at least a portion of a length of the endoscope sheath.

19) The endoscope sheath of claim 16, wherein the pattern is located towards the proximal end of the endoscope sheath.

20) The endoscope sheath of claim 16, wherein the pattern is circles, lines, squares, continuous, intermittent, spaced apart from other patters, along a portion of the endoscope sheath, or a combination thereof.
